(19) United States
(12) Patent Application Publication
(10) Pub. No.: US 2014/0249122 A1
(43) Pub. Date: Sep. 4, 2014

(54) ANTI-DIABETIC AMINOSTEROID DERIVATIVES

(71) Applicants: UNIVERSITE NICE SOPHIA ANTIPOLIS, Nice (FR); UNIVERSITE D'AUX-MARSEILLE, Marseille (FR); INSERM (Institut National de la Sante Et de la Recherche Medicale), Paris (FR); CENTRE NATIONAL DE LA RECHERCHE SCIENTIQUE, Paris (FR)

(72) Inventors: Roland Marinus Theodorus Govers, Marseille (FR); Jean-Michel Brunel, Marseille (FR)

(73) Assignees: UNIVERSITE NICE SOPHIA ANTIPOLIS, Nice (FR); UNIVERSITE D'AIX-MARSEILLE, Marseille (FR); INSERM (Institut National de la Sante Et de la Recherche Medicate), Paris (FR); CENTRE NATIONAL DE LA RECHERCHE SCIENTIFQUE, Paris (FR)

(21) Appl. No.: 14/352,220

(22) PCT Filed: Oct. 16, 2012
(86) PCT No.: PCT/FR2012/052359

§ 371 Date: Apr. 16, 2014

(30) Foreign Application Priority Data
Oct. 17, 2011 (FR) 1159377

(51) Int. Cl. C07J 43/00; C07J 41/00

(52) U.S. Cl. CPC C07J 43/003; C07J 41/0005
USPC 514/176; 552/515; 514/182; 540/106; 552/521; 514/169

(57) ABSTRACT

The present invention relates to novel aminosteroid derivatives substituted in position 3 andor 6, and to the use thereof in the context of the treatment of type 2 diabetes and of insulin resistance.

The present invention relates to novel aminosteroid derivatives, and to the use thereof in the context in particular of the treatment of type 2 diabetes and of insulin resistance.

### TECHNOLOGICAL BACKGROUND OF THE INVENTION

The prevalence of type 2 diabetes (T2D) is extremely high in our society and continues to increase at an alarming rate worldwide (175 million during the year 2000, and 350 million estimated for 2030). Associated with obesity, with a poor diet and with a lack of exercise, it should become the predominant disease in a few decades. Because of the numerous health complications and associated financial costs, this metabolic disease has become a very substantial financial burden for society, requiring the development of new anti-diabetic medicaments.

The very first symptom of type 2 diabetes is the desensitization to insulin of the liver, the skeletal muscles and the adipose tissue. The increase in blood insulin levels (as after a meal) then no longer enables sufficient uptake of sugar by the muscles and the adipose cells, nor the arrest of hepatic production of sugar. This process, called insulin resistance, is the first step in the development of hyperglycaemia. Skeletal muscle and adipose tissue are the main tissues responsible for storing blood sugar after a meal, and the GLUT4 glucose transporter in these tissues is responsible for the uptake of blood sugar. The complications associated with type 2 diabetes are severe (blindness, kidney failure, cardiac diseases), and can result in the death of the patient.

Among the molecules used in the treatment of type 2 diabetes, thiazolidinediones improve the insulin sensitivity of muscle and adipose tissue, but have considerable side effects (oedema, weight gain, and cardiac problems). Another therapeutic approach consists in administering insulin. The major drawback of insulin is that it can only be administered by injection. In addition, some patients become insensitive to insulin administrations. Other therapeutic approaches use analogues of glucagon-like peptide-1 (GLP-1; such as exenatide) and amylin mimetics (such as pramlintide). The targets of these therapies are the pancreas and the brain, but not muscle or adipose tissue. These therapies increase the blood insulin level through stimulation of insulin production by the pancreas, but can, in the long term, have an apoptotic effect with respect to the beta cells of the pancreas. Aminosteroid derivatives, in particular trodusquemine, have also been proposed, in particular for reducing obesity.

Squalamine, a steroid substituted in positions 3, 7 and 24, isolated from the shark, was initially described for its antibiotic properties (U.S. Pat. No. 5,192,756) and antiangiogenic properties (cf. patents U.S. Pat. No, 5,733,899 and U.S. Pat. No. 5 ,721,226). The formula of squalamine is the following:

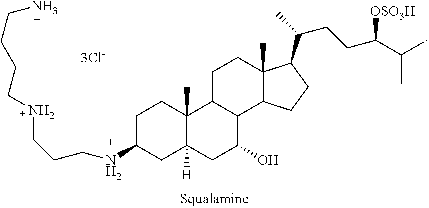

Several aminosteroids substituted in positions 3, 7 and 24 have also been described, including in particular trodusquemine, of forumla

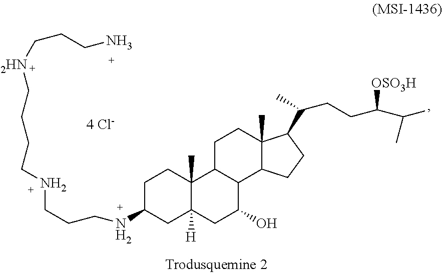

proposed for treating obesity and diabetes (cf. patent application US20090105204).

### SUMMARY OF THE INVENTION:

The inventors now propose a novel family of aminosteroid derivatives substituted in positions 3 and 6, which show a cellular sugar uptake effect and can therefore be used in particular for the treatment of type 2 diabetes and of insulin resistance.

The present invention thus provides aminosteroid derivatives of formula (I):

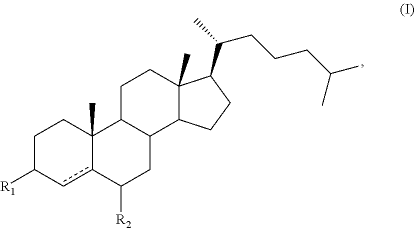

R₁ and R₂ being as defined below.

The preferred compounds are 6β-(spermine)cholestan-3β-ol and 6β-(spermidine)cholesten-3β-ol, preferably in hydrochloride form.

The invention is directed towards the compounds described herein, as medicaments.

Another subject of the invention is therefore a pharmaceutical composition comprising an aminosteroid derivative of formula (I) and a pharmaceutically acceptable carrier.

Such a composition is particularly of use in the treatment of type 2 diabetes, for reducing hyperglycaemia and complications thereof, and in the treatment of insulin resistance.

### DESCRIPTION OF THE FIGURES

FIG. 1A is a graph which shows the measurement of GLUT4 at the surface of the plasma membrane (PM), as a function of time, in the presence of insulin (100 nM), of ST10 (50 μM), or of the two combined.

FIG. 1B is a graph which shows the increase in GLUT4 at the surface of the plasma membrane (PM), as a function of the concentration of ST10 compound.

FIG. 1C is a histogram showing that the increase in GLUT4 at the surface of the plasma membrane (PM) is greater in the presence of ST10 than in the presence of trodusquemine (MSI).

FIG. 1D presents the percentage of GLUT4 at the surface of the cells as a function of the aminosterol under consideration, and of the presence (black bars) or absence (white bars) of insulin. The dashed lines represent the values observed without aminosterol, in the presence or absence of insulin.

FIG. 2 is a graph which shows that the glucose uptake in adipocytes is increased by ST10 for all the insulin concentrations tested.

FIG. 3A is a graph which shows the effect of insulin and of ST10 on the amount of GLUT4 on the plasma membrane. FIG. 3B is a conversion of FIG. 3A where the values are expressed as a function of the relative difference between the minimum and maximum signals. This figure shows that ST10 also increases the sensitivity of the cells to insulin.

FIG. 4 is a graph which shows the effect of ST10 on the amount of GLUT4 on the plasma membrane in insulin-resistant cells, compared with the effect in insulin-sensitive cells. The black bars correspond to the presence of ST10 and the white bars to the absence thereof.

FIG. 5 is a graph which shows the in vivo effect of a treatment with ST20 on the blood glucose level of mice, after injection of a dose of glucose (Glucose Tolerance Test, GTT).

FIG. 6 is a graph which shows the in vivo effect of a treatment with ST20 on the blood glucose level of mice, after injection of a dose of insulin (Insulin Tolerance Test, ITT).

FIG. 7 is a graph which shows the in vivo effect of a treatment with ST20 on the blood glucose level of obese mice, after injection of a dose of glucose (GTT).

FIGS. 8A and 8B are graphs which show the in vivo effect of a treatment with ST20 on the food intake and the weight gain of obese mice. The treatment began on day 0.

### DETAILED DESCRIPTION OF THE INVENTION

The present invention relates to novel aminosteroid derivatives of formula (I):

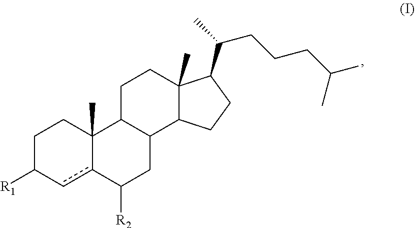

in which:

R₁ is chosen from a hydroxyl group and a polyamino chain of formula —NR₃R₄, with
R₃=−(A-X)ₚ-A-NR₆R₇, where
   each A, which may be identical or different, is an alkyl chain comprising 1 to 7 carbons, each carbon being independently optionally substituted with at least one alkyl, aryl or ester group,
   each X, which may be identical or different, is an oxygen atom, an NR_{S} group or a single bond,
   each of the R₅ is independently chosen from a hydrogen atom, an alkyl group, an aryl group or an ester group,
   R₆ and R, are independently chosen from a hydrogen atom, an alkyl group, an aryl group and an ester group,
   alternatively, the NR₆R, group can represent a nitrogenous heterocycle,
   p is an integer chosen between 1 and 4 (inclusive),
R₄ is chosen from a hydrogen atom, an alkyl group, an aryl group and an ester group,
R₂ has the same definition as R₁, R₁ and R₂ being chosen independently of one another, and
at least one of R₁ and R₂ is a polyamino chain of formula —NR₃R₄ as defined above.

The bond in the form of a dashed line denotes either a single bond or a double bond.

The above formula describes compounds which can comprise several A groups and several X groups. As explained by the expression “which may be identical or different”, each A (respectively X) group is chosen independently.

The present invention also includes the optical and geometric isomers of the derivatives of formula (I) at the level of the atoms of which the geometry is not fixed in formula (I), the racemates thereof, the tautomers thereof, the pharmaceutically acceptable salts thereof, the hydrates thereof and the mixtures thereof.

The derivatives of formula (I) defined as above which have a sufficiently acidic function or a sufficiently basic function, or both, can include the corresponding pharmaceutically acceptable salts of an organic or inorganic acid or of an organic or inorganic base.

In particular, the derivatives of formula (I) can have basic nitrogen atoms which can be monosalified or disalified with organic or inorganic acids.

The expression “pharmaceutically acceptable salts” refers to the inorganic and organic, relatively non-toxic, acid addition salts, and the base addition salts, of the compounds of the present invention. These salts can be prepared in situ during the final isolation and the purification of the compounds. In particular, the acid addition salts can be prepared by separately reacting the purified compound in its purified form with an organic or inorganic acid and by isolating the salt thus formed. Among the examples of acid addition salts are the hydrobromide, hydrochloride, sulphate, bisulphate, phosphate, nitrate, acetate, oxalate, valerate, oleate, palmitate, stearate, laurate, borate, benzoate, lactate, tosylate, citrate, maleate, fumarate, succinate, tartrate, naphthylate, mesylate, glucoheptanate, lactobionate, sulphamate, malonate, salicylate, propionate, methylenebis-b-hydroxynaphthoate, gentisic acid, isethionate, di-p-toluoyl tartrate, methanesulphonate, ethane-sulphonate, benzenesulphonate, p-toluenesulphonate, cyclohexyl sulphamate and quinateslauryl sulphonate salts, and analogues (see, for example, S.M. Berge et al. “Pharmaceutical Salts” J. Pharm. Sci, 66: p.1-19 (1977)). The acid addition salts can also be prepared by separately reacting the purified compound in its acid form with an organic or inorganic base and by isolating the salt thus formed. The acid addition salts comprise the amino and metal salts. The suitable metal salts comprise the sodium, potassium, calcium, barium, zinc, magnesium and aluminium salts. The sodium and potassium salts are preferred. The inorganic base addition salts which are suitable are prepared from metal bases which comprise sodium hydride, sodium hydroxide, potassium hydroxide, calcium hydroxide, aluminium hydroxide, lithium hydroxide, magnesium hydroxide and zinc hydroxide. The amine base addition salts which are suitable are prepared from amines which have sufficient alkalinity to form a stable salt, and preferably comprise the amines which are often used in medicinal chemistry owing to their low toxicity and their acceptability for medical use: ammonia, ethylene-diamine, N-methylglucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, tetramethylammonium hydroxide, triethylamine, dibenzylamine, ephenamine, dehydroabietylamine, N-ethylpiperidine, benzylamine, tetramethylammonium, tetra-ethylammonium, methylamine, dimethylamine, trimethylamine, ethylamine, base amino acids, for example lysine and arginine, and dicyclohexylamine, and analogues.

According to one embodiment of the invention, R₁ is a hydroxyl group.

According to another embodiment of the invention, R₂ is a polyamino chain of formula —NR₃R₄ as defined above.

According to another embodiment of the invention, each X, which may be identical or different, is an oxygen atom or an NR₅ group.

According to another embodiment of the invention, all the X of the polyamino chain are NR₅ groups.

According to another embodiment of the invention, R₆ and R, are independently chosen from a hydrogen atom, an aryl group and an ester group.

According to another embodiment of the invention, the derivatives are such that, if the bond in the form of a dashed line is a single bond and p=1, then X is a single bond.

According to other embodiments of the invention, p is 1, 2, 3 or 4.

According to one more preferred embodiment of the invention, the derivative of formula (I) is chosen from:

6β-(1,2-diaminoethane)cholestan-3β-ol ST3,
6β-(1,3-diaminopropane)cholestan-3β-ol ST4,
6β-(1,4-diaminobutane)cholestan-3β-ol ST5,
6β-(1,5-diaminopentane)cholestan-3β-ol ST6,
6β-(1,6-diaminohexane)cholestan-3β-ol ST7,
6β-(1,8-diaminooctane)cholestan-3β-ol ST8,
6β-(1,10-diaminodecane)cholestan-3β-ol ST9,
6β-(spermine)cholestan-3β-ol ST10,
6β-(1,4-bis(3-aminopropoxy)butane)cholestan-3β-ol ST11,
6β-(1,12-diaminododecane)cholestan-3β-ol ST12,
6β-(1-(3-aminopropyppyrrolidinone)cholestan-3β-ol ST14,
6β-(1-(3-aminopropyl)morpholine)cholestan-3β-ol ST15,
6β-(1-(3-aminopropyppyrrolidine)cholestan-3β-ol ST16,
6β-(1-(3-aminopropypimidazole)cholestan-3β-ol ST17,
6β-(1-(2-aminoallyppiperazine)cholestan-3β-ol ST18,
6β-(spermine)cholesten-3β-ol ST19,
6β-(spermidine)cholesten-3β-ol ST20,
3β,6β-bis(pentanediamine)cholest-3-ene ST21,
3β,6β-bis(hexanediamine)cholest-3-ene ST22,
3β,6β-bis(heptanediamine)cholest-3-ene ST23, and
3β,6β-bis(octanediamine)cholest-3-ene ST24.

According to a more preferred embodiment of the invention, the derivative of formula (I) is 6β-(spermine)cholestan-3β-ol or 6β-(spermidine)cholesten-3β-ol, having the respective formulae:

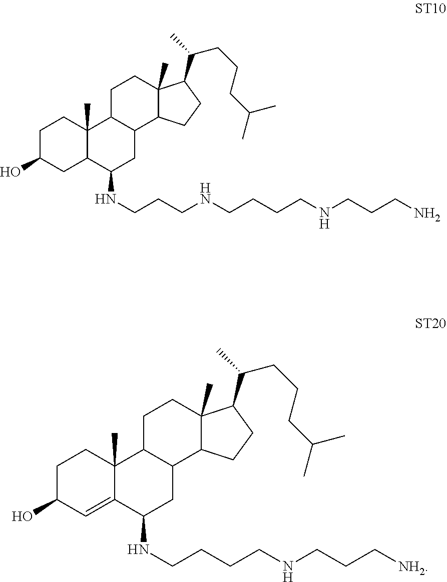 [SMILES: CC(C)CCC[C@@H](C)[C@H]1CCC2C3C[C@@H](NCCCNCCCCNCCCN)C4C[C@@H](O)CC[C@]4(C)C3CC[C@@]21C ; CC(C)CCC[C@@H](C)[C@H]1CCC2C3C[C@@H](NCCCCNCCCN)C4=C[C@@H](O)CC[C@]4(C)C3CC[C@@]21C]

These two compounds are denoted respectively “ST10” and “ST20” in the description and examples below.

In the present invention, the term “alkyl group” denotes a linear, branched or cyclic, saturated C₁-C₈, preferably C₁-C₄, hydrocarbon-based group, such as methyl, ethyl, propyl, isopropyl, butyl, isobutyl, tert-butyl, pentyl, neopentyl, n-hexyl, n-octyl. The alkyl groups can optionally have one or more substituents chosen in particular from a halogen atom, a hydroxyl group, an amino group, an alkoxyl (—O-alkyl) group, a thiol group, a thioether (—S-alkyl) group, a nitro group, a cyano group, a sulphuric (O—SO₃H) group and an ester (—CO₂-alkyl) group.

In the present invention, the term “aryl group” denotes a monocyclic, bicyclic or tricyclic aromatic hydrocarbon-based group, optionally interrupted with at least one heteroatom, in particular O, S andor N. Preferentially, the aryl group is a monocyclic or bicyclic aromatic hydrocarbon-based system having from 6 to 18 carbon atoms, even more preferentially 6 carbon atoms. Mention may be made, for example, of phenyl, naphthyl and biphenyl groups. When they are interrupted with heteroatoms, the aryl groups include pyridyl, imidazoyl, pyrrolyl and furanyl rings. The aryl groups may optionally have one or more substituents, chosen in particular from a halogen atom, an alkyl group as defined above, or an alkoxyl (—O-alkyl), thiol, thioether (—S-alkyl), hydroxyl, nitro, cyano and ester (—CO₂-alkyl) radical.

In the present invention, the term “nitrogenous heterocycle” denotes an alkyl ring comprising one or more heteroatoms chosen from N, O and S, comprising 3 to 7 ring members, optionally comprising one or more double or triple bonds, and optionally comprising one or more substituents chosen in particular from a halogen atom, a hydroxyl group, an amino group and a carbonyl (═O) group. Mention may be made, for example, of pyrrolidine, pyrrolidone, morpholine, imidazole and piperazine heterocycles.

The term “halogen atom” denotes a chlorine, bromine, iodine or fluorine atom.

There are various synthesis routes for obtaining the compounds according to the invention. The preferred preparation process calls upon a reaction for reductive amination, with titanium, of the corresponding ketosteroids under mild conditions (ambient temperature and atmospheric pressure) as illustrated below.

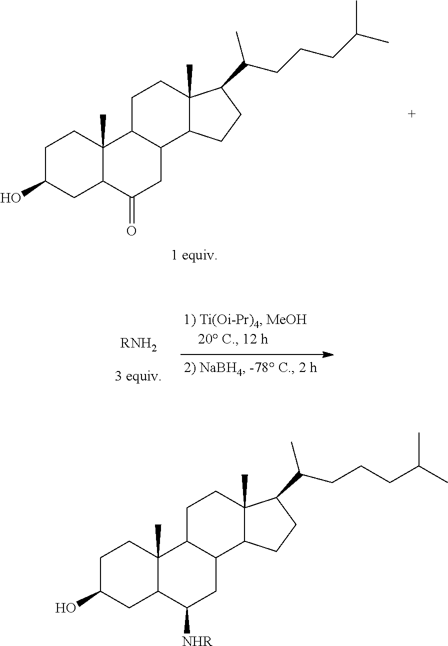

It has been shown that it is possible to significantly increase the action of insulin on the GLUT4 glucose transporter in adipocyte cell models using compounds according to the invention. The action of the aminosteroid derivatives according to the invention on the GLUT4 glucose transporter is accompanied by an increase in glucose uptake. Furthermore, this pro-insulinic action is maintained in cells made insulin resistant in vitro and in vivo. On the basis of the results obtained in mice, the inventors propose using this family of compounds, in particular the ST20 compound, to reduce the blood glucose level more rapidly in healthy individuals (without excess weight) as it did in mice. Furthermore, this faster decrease in the blood glucose level indicates faster penetration of the glucose into the cells. In muscle cells, which need glucose in order to operate efficiently, this faster provision of glucose may enable better performance levels.

In addition, the inventors have been able to show a reduction in the blood glucose level in a prolonged manner. The compounds of the invention are therefore of use for providing better control of the blood glucose level of an individual. Finally, the compounds of the invention are of use for reducing insulin resistance.

The present invention also relates to a pharmaceutical composition comprising an aminosteroid derivative as defined above and a pharmaceutically acceptable carrier.

The compounds or compositions according to the invention can be administered in various ways and in various forms. Thus, they can be administered systemically, by oral administration, by inhalation or by injection, for instance intravenously, intramuscularly, subcutaneously, transdermally, intra-arterially, etc., intravenous, intramuscular, subcutaneous and oral administration and administration by inhalation being preferred. For the injections, the compounds are generally conditioned in the form of liquid suspensions, which can be injected by means of syringes or of infusions, for example. In this regard, the compounds are generally dissolved in buffered, isotonic, physiological, etc., saline solutions which are compatible with pharmaceutical use and known to those skilled in the art. Thus, the compositions may contain one or more agents or carriers chosen from dispersants, solubilizing agents, stabilizers, preservatives, etc. Agents or carriers which can be used in liquid andor injectable formulations are in particular methylcellulose, hydroxymethylcellulose, carboxymethylcellulose, polysorbate 80, mannitol, gelatin, lactose, vegetable oils, acacia, etc.

The compounds can also be administered in the form of gels, oils, tablets, suppositories, powders, gel capsules, capsules, aerosols, etc., optionally by means of galenical forms or devices which provide prolonged andor delayed release. For this type of formulation, an agent such as cellulose, carbonates or starches is advantageously used.

It is understood that the flow rate andor the dose administered can be adjusted by those skilled in the art according to the patient, to the pathological condition concerned, to the mode of administration, etc. Typically, the compounds are administered at doses which can range between 0.1 μg and 100 mg/kg of body weight, more generally from 0.1 to 20 mg/kg, typically between 1 and 10 mg/kg. For chronic treatments, delayed or prolonged systems can be used.

The invention also relates to a method for treating type 2 diabetes or insulin resistance, by administering, to a subject suffering from such a pathological condition, an effective amount of one of the compounds according to the invention.

Preferably, this involves a subject who has become insensitive to insulin.

The compounds according to the invention are also of use for treating hyperglycaemia (namely reducing or preventing the occurrence of hyperglycaemia), or for the prevention or treatment of a complication of hyperglycaemia.

Said complications include, in particular, retinopathies, neuropathies, nephropathies, cardiovascular injuries, and lesions on the feet (diabetic foot).

The compounds of the invention are also of use for reducing the weight of an individual, in particular an overweight individual, preventing weight gain, or preventing or treating obesity.

The compounds of the invention are also of use as appetite reducers or hunger suppressants.

Finally, the compounds of the invention are of use for improving the physical performance levels of an individual, in particular via their action promoting rapid penetration of sugar into the cells.

In the context of the invention, the term “treatment” denotes preventive, curative or palliative treatment, and also the management of patients (reduction of suffering, improvement of lifespan, slowing of disease progression, etc.). The compound of the invention can be administered as sole active ingredient, or as sole anti-diabetic, or in combination with other active ingredients, in particular with other anti-diabetics. The treatment can thus be carried out in combination with other chemical or physical agents or treatments. The compounds according to the invention can therefore be conditioned and administered in a combined, separate or sequential manner with respect to other therapeutic agents or treatments. The treatments and medicaments of the invention are quite particularly intended for human beings.

A subject of the present invention is also the use of at least one compound as defined above, for the preparation of a pharmaceutical composition intended for treating type 2 diabetes or one of the pathological conditions mentioned above.

Other aspects and advantages of the present application will emerge on reading the examples which follow, which should be considered to be non-limiting illustrations.

### EXAMPLES

### Example 1

### Synthesis of the Compounds of the Invention

### I—Synthesis of the 6-aminosteroids ST3-ST18

The aminosteroids were all produced according to the same procedure.

In a two-necked round-bottomed flask placed under argon, 3 equivalents of amine under consideration (0.69×10⁻³ mol) are dissolved in 5 ml of MeOH, and then 87 μl of Ti(Oipr)₄ (0.30×10⁻³ mol) are added. After stirring for 2 minutes, 100 mg of 6-ketocholestanol (0.23×10⁻³ mol) are added to the mixture. After stirring for 24 hours, the round-bottomed flask is placed at −78° C., and then 11 mg of NaBH₄ (0.23×10⁻³ mol) are added. Two hours later, 1 ml of water is added in order to stop the reaction. Five minutes later, the mixture is filtered through a sintered glass funnel and celite®. The filtrate is evaporated under a strong vacuum. The product is purified by silica gel chromatography (eluent: CH₂Cl₂MeOHNH₄OH (7/3/1)).

### 6β-(1,2-diaminoethane)cholestan-3β-ol ST3

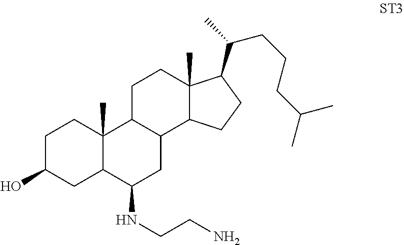 [SMILES: CC(C)CCC[C@@H](C)[C@H]1CCC2C3C[C@@H](NCCN)C4C[C@@H](O)CC[C@]4(C)C3CC[C@@]21C]

Yield: 96%. ¹HNMR: δ=3.29-3.63 (m, 1H), 0.57-2.83 (m, 53H); ¹³C NMR: δ=71.57, 58.79, 58.61, 56.28, 56.00, 54.74, 50.95, 47.27, 42.62, 41.88, 39.93, 39.48, 39.00, 36.23, 36.14, 36.05, 35.75, 35.64, 31.54, 30.39, 27.96, 24.36, 23.79, 22.76, 22.52, 21.03, 18.63, 15.21, 12.12. C₂₉H₅₄N₂O; MS (ESI) mz=447.3 [M+H]⁺

### 6β-(1,3-diaminopropane)cholestan-3β-ol ST4

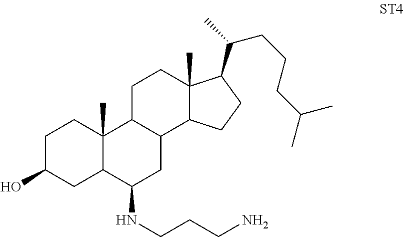 [SMILES: CC(C)CCC[C@@H](C)[C@H]1CCC2C3C[C@@H](NCCCN)C4C[C@@H](O)CC[C@]4(C)C3CC[C@@]21C]

Yield: 63%. ¹H NMR: δ=0.66-3.61 (m, 56H); ¹³C NMR: δ=71.60, 58.78, 56.28, 56.02, 54.80, 47.32, 46.91, 42.60, 39.94, 39.48, 38.95, 36.14, 35.99, 35.75, 35.64, 31.57, 30.43, 28.17, 27.95, 24.36, 23.77, 22.76, 22.51, 21.02, 18.63, 16.04, 12.06. C₃₀H₅₆N₂O; MS (ESI) mz=461.3 [M+H]⁺

### 6β-(1,4-diaminobutane)cholestan-3β-ol ST5

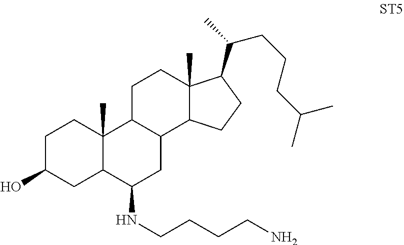 [SMILES: CC(C)CCC[C@@H](C)[C@H]1CCC2C3C[C@@H](NCCCCN)C4C[C@@H](O)CC[C@]4(C)C3CC[C@@]21C]

Yield: 73%. ¹H NMR: δ=0.66-3.57 (m, 58H); ¹³C: δ=71.65, 59.88, 58.54, 56.29, 56.04, 54.75, 48.18, 47.29, 42.71, 42.64, 39.94, 39.50, 39.04, 36.16, 35.78, 35.65, 31.56, 31.03, 30.40, 29.67, 27.99, 25.96, 24.35, 23.81, 22.79, 22.54, 21.05, 18.65, 16.33, 14.09, 12.15. C₃₁H₅₈N₂O; MS (ESI) mz=475.4 [M+H]⁺

### 6β-(1,5-diaminopentane)cholestan-3β-ol ST6

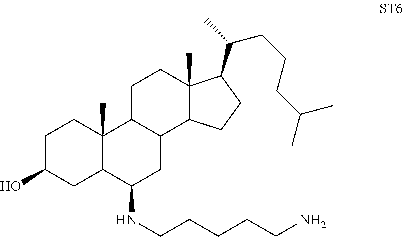 [SMILES: CC(C)CCC[C@@H](C)[C@H]1CCC2C3C[C@@H](NCCCCCN)C4C[C@@H](O)CC[C@]4(C)C3CC[C@@]21C]

Yield: 90%. ¹H NMR: δ=0.65-3.61 (m, 60H); ¹³C: δ=71.76, 60.05, 58.76, 56.54, 56.29, 56.10, 54.85, 48.76, 47.34, 42.70, 42.62, 40.58, 39.98, 39.49, 38.92, 36.15, 35.76, 35.63, 35.28, 31.03, 30.40, 30.21, 28.19, 27.87, 25.93, 24.34, 23.79, 22.78, 22.52, 21.05, 18.64, 12.12. C₃₂H₆₀N₂O; MS (ESI) mz=489.5 [M+H]⁺

### 6β-(1,6-diaminohexane)cholestan-3β-ol ST7

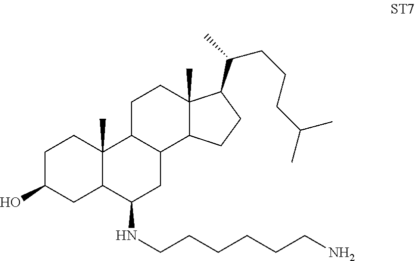 [SMILES: CC(C)CCC[C@@H](C)[C@H]1CCC2C3C[C@@H](NCCCCCCN)C4C[C@@H](O)CC[C@]4(C)C3CC[C@@]21C]

Yield: 29%. ¹H NMR: δ=3.30-3.65 (m, 1H), 0.66-2.59 (m, 61H); ¹³C: δ=71.72, 58.73, 56.30, 56.12, 54.86, 48.72, 47.35, 42.62, 39.99, 39.49, 38.93, 36.34, 36.15, 36.01, 35.77, 35.63, 31.59, 30.40, 30.27, 28.19, 27.97, 27.10, 24.33, 23.79, 22.77, 22.52, 21.05, 18.64, 16.20, 12.12. C₃₃H₆₂N₂O; MS (ESI) mz=503.4 [M+H]⁺

### 6β-(1,8-diaminooctane)cholestan-3β-ol ST8

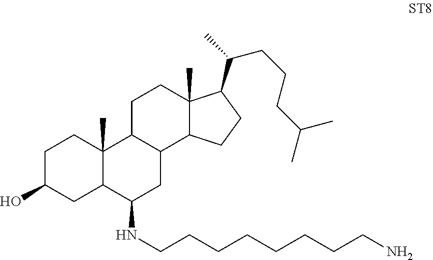 [SMILES: CC(C)CCC[C@@H](C)[C@H]1CCC2C3C[C@@H](NCCCCCCCCN)C4C[C@@H](O)CC[C@]4(C)C3CC[C@@]21C]

Yield: 32%. ¹H NMR: δ=3.18-3.63 (m, 2H), 0.61-2.67 (m, 64H); ¹³C: δ=71.68, 58.73, 56.28, 56.11, 54.86, 48.73, 47.35, 42.60, 39.97, 39.47, 38.93, 36.30, 36.13, 36.04, 35.75, 35.61, 31.56, 30.38, 30.19, 29.43, 29.35, 29.25, 28.17, 27.95, 27.13, 26.71, 24.31, 23.77, 22.76, 22.50, 21.03, 18.63, 16.19, 12.10. C₃₅H₆₆N₂O; MS (ESI) mz=531.5 [M+H]⁺

### 6β-(1,10-diaminodecane)cholestan-3β-ol ST9

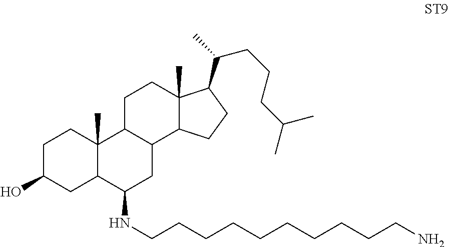 [SMILES: CC(C)CCC[C@@H](C)[C@H]1CCC2C3C[C@@H](NCCCCCCCCCCN)C4C[C@@H](O)CC[C@]4(C)C3CC[C@@]21C]

Yield: 68%. ¹³C NMR: δ=71.49, 60.06, 58.73, 56.50, 56.24, 56.07, 54.85, 48.77, 48.21, 47.35, 42.63, 42.55, 42.09, 39.43, 36.09, 35.70, 35.59, 35.20, 33.68, 31.55, 30.98, 30.33, 29.46, 29.37, 28.14, 27.90, 27.20, 26.77, 25.88, 24.23, 23.73, 22.71, 22.47, 21.00, 18.59, 16.15, 12.06. C₃₇H₇₀N₂O; MS (ESI) mz=559.5 [M+H]⁺

### 6β-(spermine)cholestan-3β-ol ST10

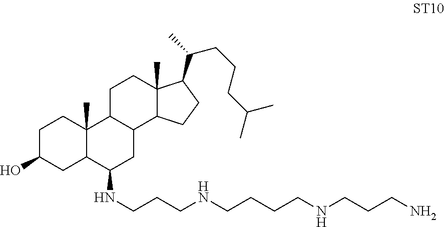 [SMILES: CC(C)CCC[C@@H](C)[C@H]1CCC2C3C[C@@H](NCCCNCCCCNCCCN)C4C[C@@H](O)CC[C@]4(C)C3CC[C@@]21C]

Yield: 24.5%. ¹³C NMR: δ=71.50, 58.96, 56.27, 56.05, 54.78, 49.98, 49.21, 47.99, 47.84, 47.34, 42.62, 40.47, 39.94, 39.49, 39.06, 36.44, 36.14, 35.86, 35.77, 35.63, 33.58, 31.61, 30.45, 28.18, 27.97, 24.37, 23.78, 22.78, 22.52, 21.03, 18.63, 16.30, 12.13. C₃₇H₇₂N₄O; MS (ESI) mz=589.5 [M+H]⁺

### 6β-(1,4-bis(3-aminopropoxy)butane)cholestan-3β-ol ST11

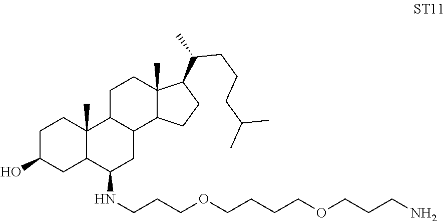 [SMILES: CC(C)CCC[C@@H](C)[C@H]1CCC2C3C[C@@H](NCCCOCCCCOCCCN)C4C[C@@H](O)CC[C@]4(C)C3CC[C@@]21C]

Yield: 98%. ¹H NMR: δ=0.47-3.92 (m, 70H); ¹³C: δ=71.14, 70.52, 69.36, 68.73, 68.68, 58.71, 56.12, 55.94, 54.71, 49.55, 47.22, 46.11, 42.44, 39.82, 39.31, 39.26, 39.18, 38.83, 35.97, 35.59, 35.46, 32.46, 32.26, 31.35, 30.21, 28.02, 27.78, 26.30, 26.24, 24.15, 23.62, 22.61, 22.36, 20.88, 18.47, 16.03, 11.95. C₃₇H₇₀N₂O₃; MS (ESI) mz=691.8 [M+H]³⁰

### 6β-(1,12-diaminododecane)cholestan-3β-ol ST12

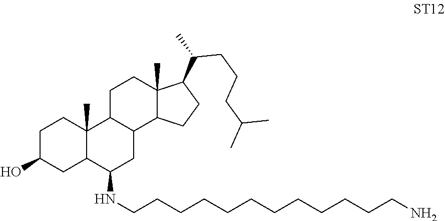 [SMILES: CC(C)CCC[C@@H](C)[C@H]1CCC2C3C[C@@H](NCCCCCCCCCCCCN)C4C[C@@H](O)CC[C@]4(C)C3CC[C@@]21C]

Yield: 15%. ¹³C NMR: δ=71.48, 60.05, 58.74, 56.49, 56.23, 56.07, 54.84, 50.02, 48.82, 48.21, 47.33, 42.64, 42.10, 40.50, 39.43, 38.92, 36.09, 35.70, 35.59, 33.70, 31.56, 30.98, 30.33, 30.29, 29.49, 29.40, 29.21, 27.91, 27.40, 26.79, 25.88, 24.26, 23.73, 22.71, 22.47, 21.00, 18.59, 12.06. C₃₉H₇₄N₂O; MS (ESI) mz=587.5 [M+H]⁺

### 6β-(1-(3-aminopropyl)pyrrolidinone)cholestan-3β-ol ST14

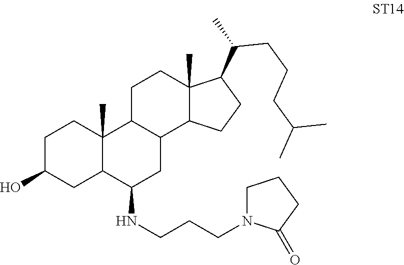 [SMILES: CC(C)CCC[C@@H](C)[C@H]1CCC2C3C[C@@H](NCCCN4CCCC4=O)C4C[C@@H](O)CC[C@]4(C)C3CC[C@@]21C]

Yield: 92%. ¹H NMR: δ=5.15-5.23 (m, 4H), 0.459-3.50 (m, 56H); ¹³C: δ=174.73, 71.33, 58.52, 56.13, 55.92, 54.69, 53.29, 47.21, 46.89, 45.65, 42.46, 40.38, 39.83, 39.43, 39.32, 38.76, 35.97, 35.60, 35.48, 31.40, 30.87, 30.77, 30.24, 28.04, 27.80, 24.19, 23.63, 22.61, 22.36, 20.88, 18.48, 17.76, 16.04, 11.96. C₃₄H₆₀N₂O₂; MS (ESI) mz=529.6 [M+H]⁺

### 6β-(1-(3-aminopropyl)morpholine)cholestan-3β-ol ST15

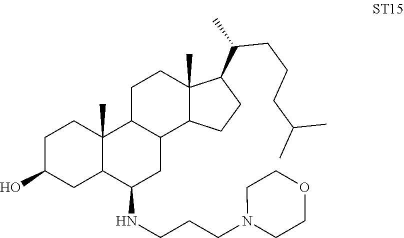 [SMILES: CC(C)CCC[C@@H](C)[C@H]1CCC2C3C[C@@H](NCCCN4CCOCC4)C4C[C@@H](O)CC[C@]4(C)C3CC[C@@]21C]

Yield: 96%. ¹H NMR: δ=5.16-5.29 (m, 2H), 3.63-3.65 (m, 6H), 0.53-2.68 (m, 54H); ¹³C: δ=71.46, 66.82, 58.77, 57.40, 56.74, 56.19, 55.96, 54.75, 53.73, 53.67, 53.29, 47.26, 42.50, 39.86, 39.37, 38.84, 36.10, 36.04, 35.55, 34.91, 31.53, 30.30, 29.55, 28.08, 27.85, 27.08, 24.25, 23.69, 22.67, 22.42, 20.94, 18.54, 16.15, 12.01. C₃₄H₆₂N₂O₂; MS (ESI) mz=531.8 [M+H]⁺

### 6β-(1-(3-aminopropyl)pyrrolidine)cholestan-3β-ol ST16

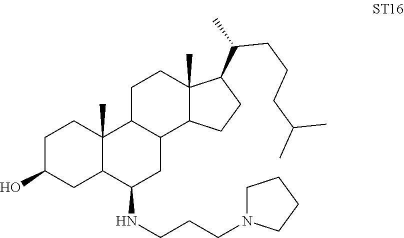 [SMILES: CC(C)CCC[C@@H](C)[C@H]1CCC2C3C[C@@H](NCCCN4CCCC4)C4C[C@@H](O)CC[C@]4(C)C3CC[C@@]21C]

Yield: 80%. ¹H NMR: δ=0.62-3.97 (m, 62H); ¹³C: δ=71.41, 58.74, 56.23, 56.01, 54.98, 54.78, 54.18, 47.59, 47.27, 42.54, 39.91, 39.41, 38.92, 36.07, 35.99, 35.89, 35.70, 35.56, 35.08, 31.47, 30.33, 29.32, 28.12, 27.88, 25.67, 24.26, 23.73, 23.29, 22.71, 22.46, 20.97, 18.57, 16.09, 12.03. C₃₄H₆₂N₂O; MS (ESI) mz=515.7 [M+H]⁺

### 6β-(1-(3-aminopropyl)imidazole)cholestan-3β-ol ST17

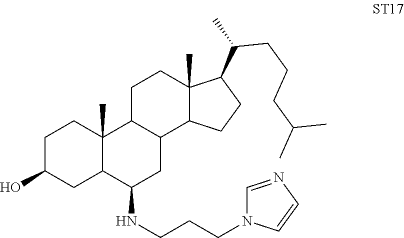 [SMILES: CC(C)CCC[C@@H](C)[C@H]1CCC2C3C[C@@H](NCCCn4ccnc4)C4C[C@@H](O)CC[C@]4(C)C3CC[C@@]21C]

Yield: 64%. ¹H NMR: δ=6.87-7.44 (m, 4H), 3.96-4.03 (m, 2H), 3.56-3.63 (m, 1H), 0.56-2.70 (m, 50H); ¹³C: δ=137.14, 128.95, 118.92, 71.35, 58.93, 56.21, 55.90, 54.67, 47.14, 44.86, 44.50, 42.56, 39.84, 39.41, 38.90, 38.52, 36.07, 35.82, 35.68, 35.61, 31.67, 31.44, 30.40, 28.10, 27.91, 24.27, 23.72, 22.72, 22.47, 20.97, 18.59, 16.27, 12.06. C₃₃H₅₇N₃O; MS (ESI) mz=512.7 [M+H]⁺

### 6β-(1-(2-aminoallynpiperazine)cholestan-3β-ol ST18

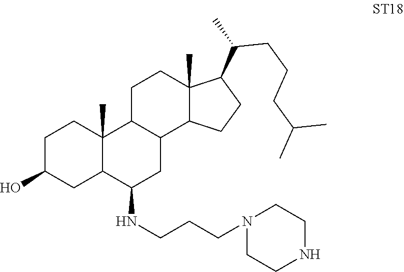 [SMILES: CC(C)CCC[C@@H](C)[C@H]1CCC2C3C[C@@H](NCCCN4CCNCC4)C4C[C@@H](O)CC[C@]4(C)C3CC[C@@]21C]

Yield: 76%. ¹H NMR: δ=0.64-4.02 (m, 61H); ¹³C: δ=71.68, 59.08, 58.12, 56.27, 56.06, 54.81, 54.07, 53.91, 53.80, 47.32, 45.91, 45.42, 42.62, 39.94, 39.46, 38.99, 36.12, 35.74, 35.61, 35.16, 31.57, 30.42, 28.17, 27.96, 25.95, 24.33, 23.77, 22.76, 22.52, 21.03, 18.64, 16.25, 12.17. C₃₃H₆₁N₃O; MS (ESI) mz=516.6 [M+H]⁺

### II—Synthesis of the aminosteroids ST19-ST20

The aminosteroids ST19-ST20 were produced according to the same procedure.

In a two-necked round-bottomed flask placed under argon, 3 equivalents of amine under consideration (2×10⁻³ mol)are dissolved in 5 ml of MeOH, and then 600 mg of Ti(Oipr)₄(2.1×10⁻³mol) are added. After stirring for 2 minutes, 250 mg of 3,6-diketocholestenone (6.28×10⁻⁴ mol) are added to the mixture. After stirring for 24 hours, the round-bottomed flask is placed at −78° C., and then 100 mg of NaBH₄ (3.3×10⁻³ mol) are added. Two hours later, 1 ml of water is added in order to stop the reaction. Five minutes later, the mixture is filtered through a sintered glass funnel and celite. The filtrate is evaporated under a strong vacuum. The product is purified by silica gel chromatography (eluent: CH₂Cl₂MeOHNH₄OH (7/3/1)).

### 6β-(spermine)cholesten-3β-ol ST19

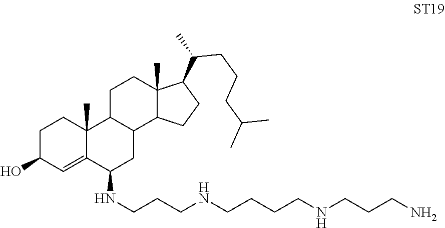 [SMILES: CC(C)CCC[C@@H](C)[C@H]1CCC2C3C[C@@H](NCCCNCCCCNCCCN)C4=C[C@@H](O)CC[C@]4(C)C3CC[C@@]21C]

Yield: 44%. ¹H NMR: δ=5.62 (s, 1H), 3.33-3.40 (m, 3H), 2.88-2.97 (m, 15H), 0.76-2.04 (m, 52H); ¹³C: δ=146.81, 128.44, 62.77, 58.06, 56.35, 48.44, 47.34, 47.25, 44.17, 41.63, 41.14, 40.77, 39.30, 38.55, 37.82, 37.56, 32.31, 31.86, 31.68, 29.73, 29.38, 27.90, 27.42, 27.30, 27.18, 26.02, 25.75, 25.41, 23.69, 23.44, 22.58, 22.04, 20.94, 19.74, 13.01, 12.94. C37H₇₀N₄O; MS (ESI) mz=586.555 [M+H]⁺

### 6β-(spermidine)cholesten-3β-ol ST20

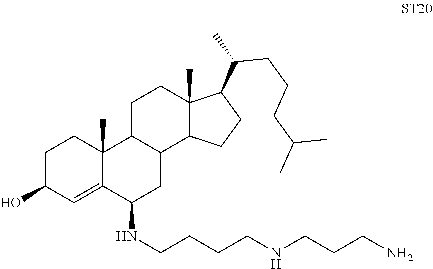 [SMILES: CC(C)CCC[C@@H](C)[C@H]1CCC2C3C[C@@H](NCCCCNCCCN)C4=C[C@@H](O)CC[C@]4(C)C3CC[C@@]21C]

Yield: 63%. ¹H NMR: δ=5.71 (s, 1H), 3.56-2.81 (m, 13H), 2.05-0.69 (m, 49H); ¹³C: δ=150.00, 118.53, 69.32, 57.62, 57.37, 56.03, 55.81, 50.22, 45.53, 43.74, 43.22, 42.35, 41.19, 40.72, 40.35, 39.13, 38.27, 37.38, 37.13, 35.73, 32.50, 30.85, 29.87, 29.28, 29.17, 27.67, 25.29, 24.98, 23.24, 23.00, 22.30, 20.29, 19.26, 12.47. C34H63N30; MS (ESI) mz=529.532 [M+H]⁺

### III—Synthesis of the aminosteroids ST21-ST24

The aminosteroids were all produced according to the same procedure.

In a two-necked round-bottomed flask placed under argon, 6 equivalents of amine under consideration (4×10⁻³ mol) are dissolved in 5 ml of MeOH, and then 1.2 g of Ti(Oipr)₄ (4.2×10⁻³ mol) are added. After stirring for 2 minutes, 250 mg of 3,6-diketocholestenone (6.28×10⁻⁴ mol) are added to the mixture. After stirring for 24 hours, the round-bottomed flask is placed at -78° C., and then 100 mg of NaBH₄ (3.3×10⁻³ mol) are added. Two hours later, 1 ml of water is added in order to stop the reaction. Five minutes later, the mixture is filtered through a sintered glass funnel and celite. The filtrate is evaporated under a strong vacuum. The product is purified by silica gel chromatography (eluent: CH₂Cl₂MeOHNH₄OH (731)).

### 3β,6β-bis(pentanediamine)cholest-3-ene ST21

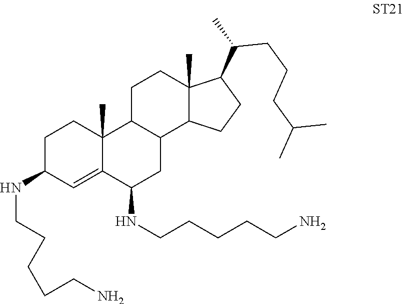 [SMILES: CC(C)CCC[C@@H](C)[C@H]1CCC2C3C[C@@H](NCCCCCN)C4=C[C@@H](NCCCCCN)CC[C@]4(C)C3CC[C@@]21C]

Yield: 54%. ¹H NMR: δ=5.51 (s, 1H), 3.53-3.40 (m, 2H), 2.75-2.15 (m, 12H), 1.91-0.41 (m, 55H); ¹³C: δ=138.86, 116.23, 66.81, 58.26, 57.31, 56.35, 54.32, 47.42, 46.53, 44.62, 42.10, 39.62, 36.32, 34.55, 31.14, 29.81, 29.41, 28.53, 27.95, 24.16, 22.14, 21.13, 18.72, 13.52. C₃₇H₇₀N₄; MS (ESI) mz=572.51 [M+H]⁺

### 3β,6β-bis(hexanediamine)cholest-3-ene ST22

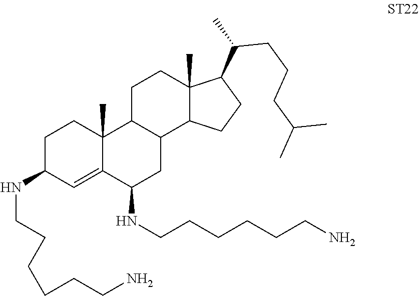 [SMILES: CC(C)CCC[C@@H](C)[C@H]1CCC2C3C[C@@H](NCCCCCCN)C4=C[C@@H](NCCCCCCN)CC[C@]4(C)C3CC[C@@]21C]

Yield: 43%. ¹H NMR: δ=5.51 (s, 1H), 4.80-4.65 (m, 4H), 3.59-2.52 (m, 8H), 1.91-0.67 (m, 61H); ¹³C: δ=138.92, 118.23, 66.88, 58.29, 57.33, 56.35, 54.36, 47.70, 46.80, 44.62, 41.80, 39.62, 39.01, 36.23, 35.70, 34.55, 33.70, 32.03, 29.61, 28.06, 26.91, 25.53, 24.27, 24.15, 22.70, 21.23, 18.73, 12.23. C₃₉H₇₄N₄; MS (ESI) mz=600.62 [M+H]⁺

### 3β,6β-bis(heptanediamine)cholest-3-ene ST23

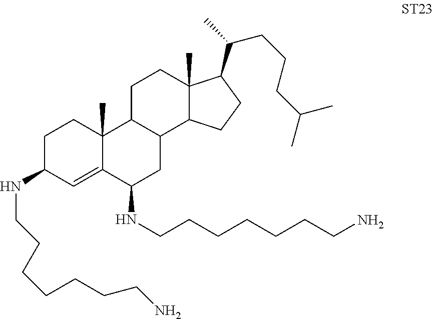 [SMILES: CC(C)CCC[C@@H](C)[C@H]1CCC2C3C[C@@H](NCCCCCCCN)C4=C[C@@H](NCCCCCCCN)CC[C@]4(C)C3CC[C@@]21C]

Yield: 51%. ¹H NMR: δ=5.52 (s, 1H), 3.53-2.07 (m, 15H), 1.93-0.62 (m, 62H); ¹³C: δ=137.34, 115.23, 67.01, 58.26, 57.31, 56.35, 54.32, 47.32, 46.53, 44.63, 42.10, 41.80, 39.52, 36.32, 34.55, 31.15, 29.71, 29.41, 28.53, 27.95, 24.13, 22.14, 21.13, 18.71, 12.52. C₄₁H₇₈N₄; MS OD mz=628.52 [M+H]⁺

### 3β,6β-bis(octanediamine)cholest-3-ene ST24

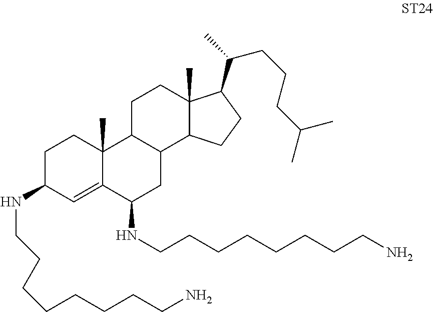 [SMILES: CC(C)CCC[C@@H](C)[C@H]1CCC2C3C[C@@H](NCCCCCCCCN)C4=C[C@@H](NCCCCCCCCN)CC[C@]4(C)C3CC[C@@]21C]

Yield: 52%. ¹H NMR: δ=5.48 (s, 1H), 3.47-2.35 (m, 16H), 2.10-0.58 (m, 65H); ¹³C: δ=139.82, 116.11, 66.81, 58.22, 57.31, 56.35, 54.32, 47.42, 46.53, 44.62, 42.49, 42.10, 39.62, 36.32, 34.55, 34.36, 31.14, 29.81, 29.41, 28.55, 27.95, 24.66, 22.14, 21.33, 19.02, 14.52. C₄₃H₈₂N₄; MS OD mz=656.62 [M+H]⁺

### Example 2

### Study of the Effect of the Compounds According to the Invention on GLUT4

One of the key components in the insulin-induced uptake of sugar by myocytes and adipocytes is the GLUT4 glucose (sugar) transporter. When insulin binds to its receptor at the surface of these cells, or during a muscle contraction, intracellular signalling pathways are activated, resulting in the translocation of GLUT4 from its intracellular storage compartment to the plasma membrane, where it enables sugar to enter from the extracellular medium. GLUT4 therefore plays an important role in carbohydrate homeostasis and, consequently, in T2D.

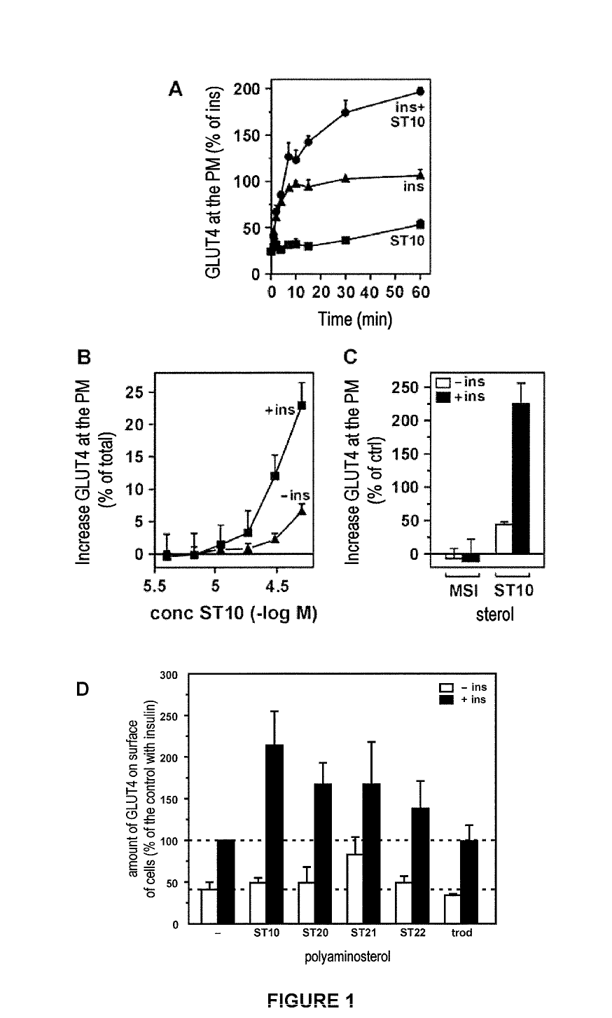

3T3-L1 adipocytes are stimulated for 20 minutes with the aminosteroids (50 μM), in the presence or absence of insulin (1 nM), and are labelled for GLUT4 at the surface of the cells. The percentage of GLUT4 at the surface of the cells is then determined. A comparison is carried out between the 6β-(spermine)cholestan-3(3-ol compound (ST10 compound) and trodusquemine (MSI-1436). The results shown in FIGS. 1A to 1C show that ST10, but not trodusquemine, increases the effectiveness of insulin on GLUT4 translocation in 3T3-L1 adipocytes. FIG. 1D shows that other compounds of the invention have an effect which is just as advantageous.

### Example 3

### Effect of the ST10 Derivative on Glucose Uptake

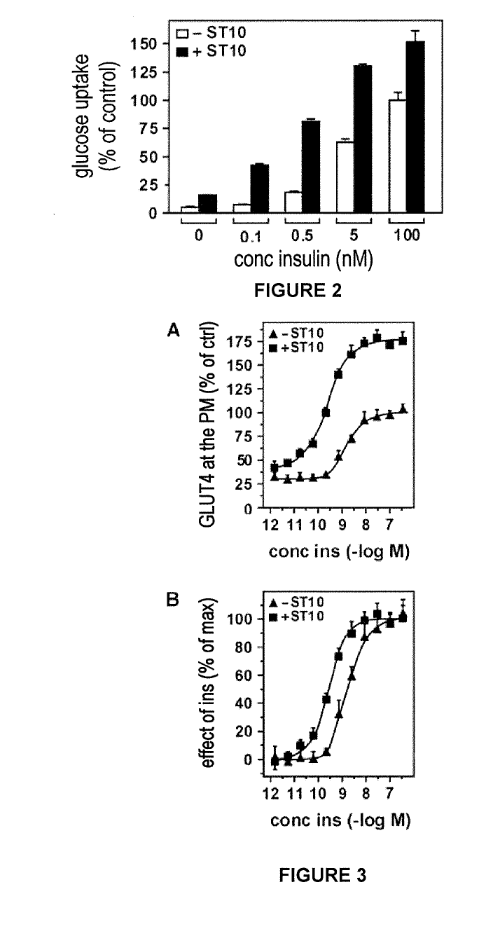

3T3-L1 adipocytes were incubated in the presence or absence of insulin, at various concentrations, and in the presence or absence of 6β-(spermine)cholestan-3(3-ol (ST10 compound). The glucose uptake was measured and expressed as percentage of the maximum uptake in the absence of aminosteroid. FIG. 2 shows the results obtained. The aminosteroid derivative according to the invention increases glucose uptake in the adipocytes.

### Example 4

### Effect of the ST10 Derivative on the Sensitivity of adipocytes to Insulin

The effect of insulin and of ST10 on the amount of GLUT4 on the plasma membrane was measured (FIG. 3A) and the sensitivity of the cells to insulin was calculated (FIG. 3B). The ED50 is reduced from 1.61 to 0.28 nM (p<0.0001).

### Example 5

### Effect of the ST10 Derivative on GLUT4 in Insulin-Resistant Cells

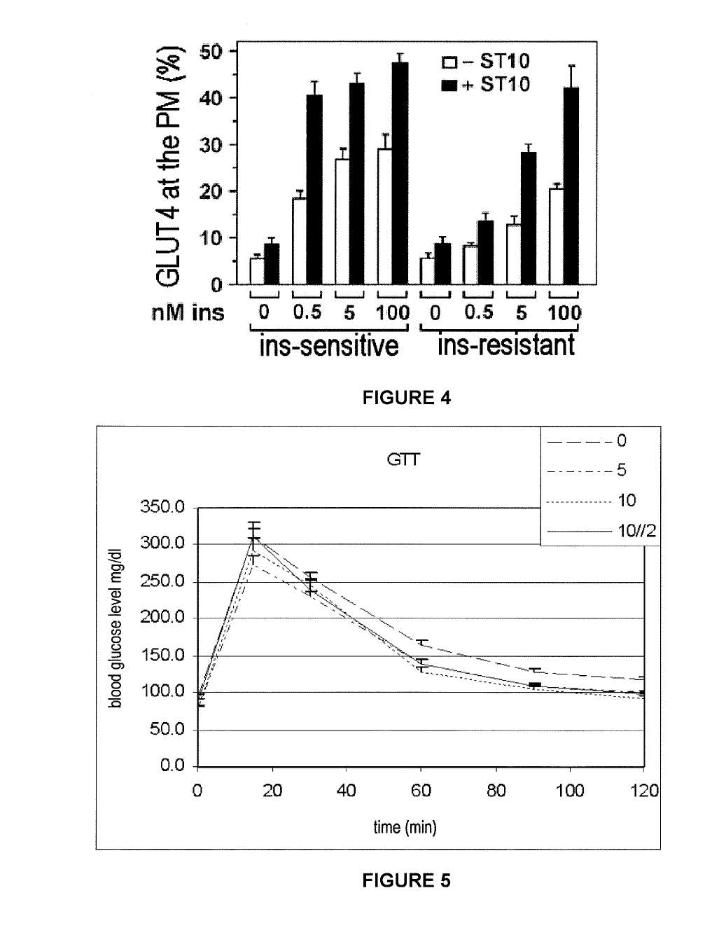

After having been made insulin resistant by treatment with insulin for 24 h, the adipocytes were stimulated for 20 min with insulin, in the presence (black bars) or in the absence (white bars) of 6β-(spermine)cholestan-3(3-ol (ST10 compound). The amount of GLUT4 on the surface of the cells was determined (FIG. 4). The insulin-resistant cells show a reduction in the action of insulin, but in these cells, aminosteroid according to the invention also increases the effect of the insulin.

### Example 6

### Effect of the ST20 Derivative on Blood Glucose Level (in vivo Test Carried out in Mice)

Glucose Tolerance Test (GTT)

Mice (thin) were treated, for two weeks, with the ST20 derivative at doses of: 0 mg/kg/day, 5 mg/kg/day, 10 mg/kg/day or 10 mg/kg every two days. A dose of glucose was injected into these mice (at t=0). The blood glucose level of the mice was measured until 120 minutes after the glucose injection. As shown in FIG. 5, the increase in blood glucose level is due to the injection of glucose and the subsequent decrease in blood glucose level is due to the action of insulin. For the three groups of mice treated with the ST20 derivative, the blood glucose level decreases more rapidly than for the group of untreated mice. The treatment with the ST20 derivative therefore potentiates the effect of insulin.

Insulin Tolerance Test (ITT)

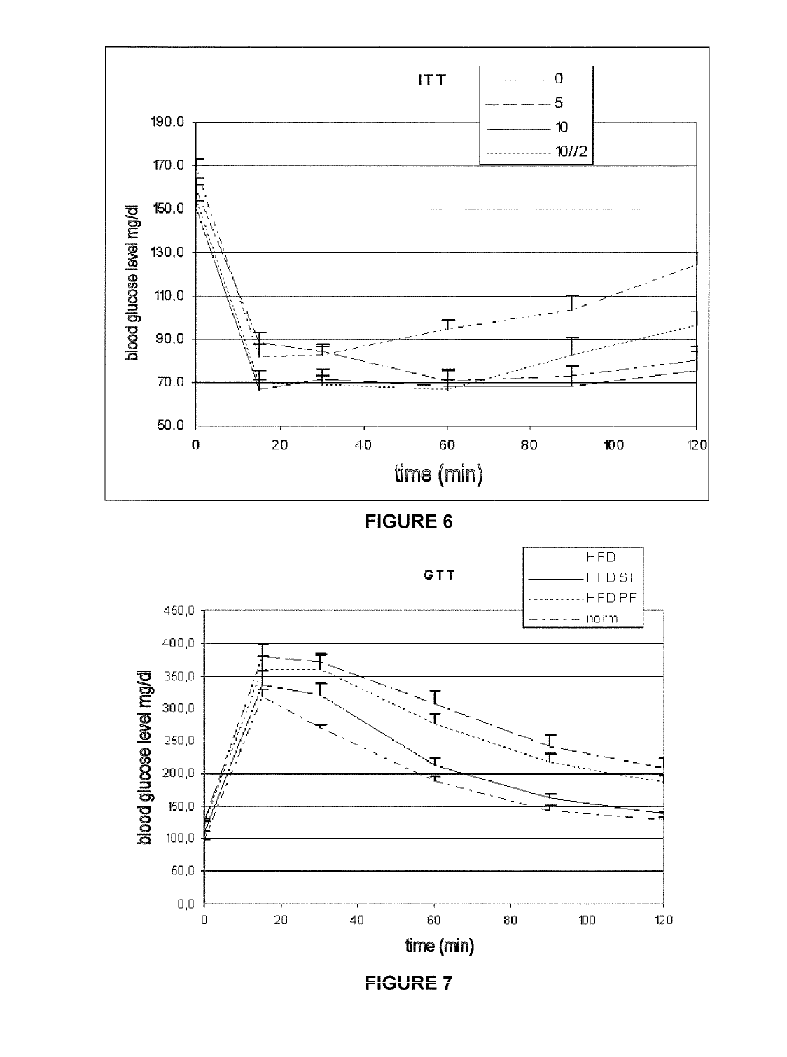

Healthy mice were treated in the same way as previously. A dose of insulin was injected into these mice (at t=0). The blood glucose level of the mice was measured until 120 minutes after the insulin injection. As shown in FIG. 6, the decrease in blood glucose level is due to the injection of insulin. For the three groups of mice treated with the ST20 derivative, the decrease in blood glucose level is prolonged over time in comparison with the blood glucose level of the group of untreated mice. The treatment with the ST20 derivative therefore prolongs the effect of insulin. Furthermore, this treatment did not cause severe hypoglycaemia in this test. This example demonstrates that ST20 reduces the blood glucose level in a prolonged manner.

### Example 7

### Effect of the ST20 Derivative on the Blood Glucose Level of Obese Mice

Glucose Tolerance Test (GTT)

Four groups of mice were formed: the HFD (high fat diet) group having followed, for 12 weeks, a diet rich in fat and with an unlimited amount of food, the HFD ST (high fat diet sterol treatment) group having followed the same diet and having been treated for one week with the ST20 derivative (10 mg/kg every two days), the HFD PF (high fat diet “pair feeding”) group having a diet equivalent to the amount of food consumed by the HFD ST group, and the norm (normal) group having a normal diet. A dose of glucose was injected into these mice (at t=0). The blood glucose level of the mice was measured until 120 minutes after the glucose injection. As shown in FIG. 7, the increase in blood glucose level is due to the injection of glucose and the subsequent decrease in blood glucose level is due to the action of insulin. The difference in blood glucose level between the HFD and norm groups clearly shows the insulin resistance of the HFD mice. The HFD ST group exhibits a decrease in blood glucose level which is significantly greater than the HFD and HFD PF groups. The treatment with the ST20 derivative is effective in the obese mice and reduces the insulin resistance.

### Example 8

### Effect of the ST20 Derivative on the Weight of the Mice

The effect of an administration of the compounds of the invention on the body mass of the mice, and the food intake, was evaluated.

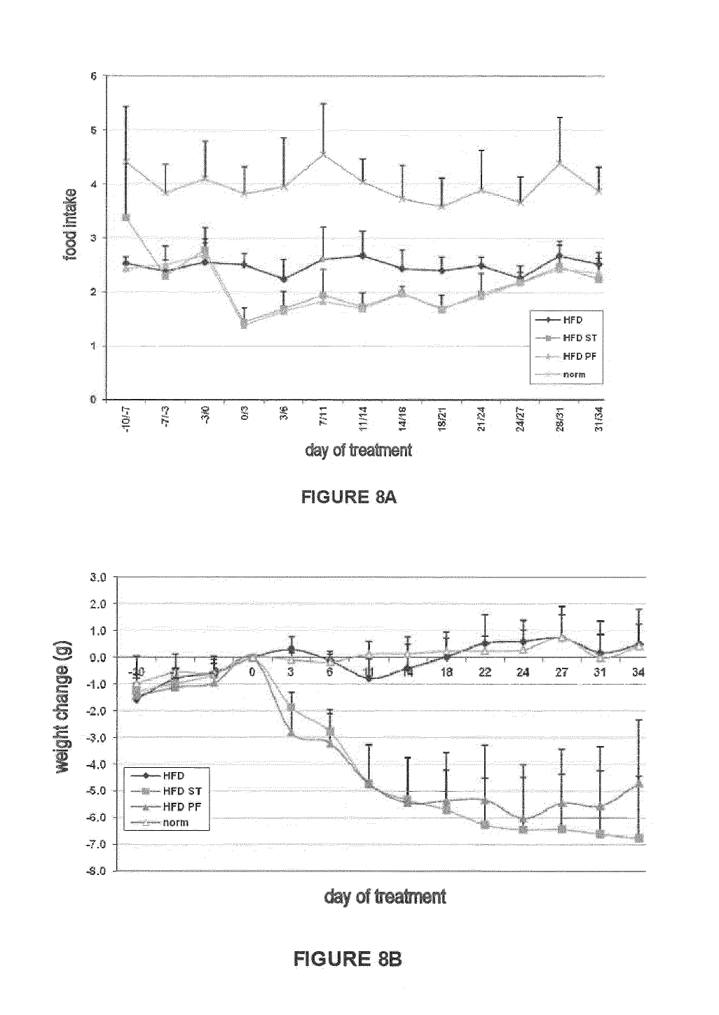

In the thin mice, the injection of the ST20 derivative (at a dose of 5 mg/kg/day, 10 mg/kg/day or 10mgday every two days) caused a transient decrease in food intake, during the first 4 days. This caused a slight reduction in the weight of the treated mice compared with the untreated mice. Groups of obese mice were formed as in Example 7. In these obese mice, the injection of the ST20 derivative (at a dose of 10 mg/kg every two days) caused a decrease in food intake (FIG. 8A), which lasted more than three weeks. The control pair feeding mice (HFD PF group) received similar amounts of food. The reduction in food intake resulted in a marked decrease in body weight (FIG. 8B), which persisted throughout the experiment (5 weeks). The body weight of the pair feeding mice (HFD PF group) decreased in a manner similar to those of the treated mice (HFD ST group), indicating that the effect of the ST20 derivative on the body weight is due to the decrease in food consumption.

In conclusion, the injection of ST20 into obese mice induces a sustained reduction of body weight due to the reduction of food intake.

1. A method of treatment of a disease in a subject in need thereof, wherein the disease is selected from the group consisting of type 2 diabetes, insulin resistance, hyperglycaemia, and a complication of hyperglycaemia, said method comprising administering to the subject a pharmaceutical composition comprising an aminosteroid derivative of formula (I) 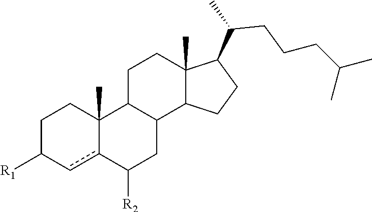
wherein:
R₁ and R₂ are each, independently, a hydroxyl group or a polyamino chain of formula —NR₃R₄;
R₃ is -(A-X)ₚ-A-NR₆R₇;
each A, which may be identical or different, is an alkyl chain comprising 1 to 7 carbons, each carbon being independently optionally substituted with at least one alkyl, aryl or ester group;
each X, which may be identical or different, is an oxygen atom, an NR₅ group or a single bond;
each of the R₅ is independently a hydrogen atom, an alkyl group, an aryl group or an ester group;
R₆ and R₇ are each, independently, a hydrogen atom, an alkyl group, an aryl group or an ester group;
or R₆ and R₇ together with the N atom to which they are attached form a heterocyclyl group;
p is; and
R₄ is a hydrogen atom, an alkyl group, an aryl group or an ester group; wherein at least one of R₁ and R₂ is a polyamino chain of formula —NR₃R₄ and a pharmaceutically acceptable carrier.

2. The method of claim 1, wherein subject is insensitive to insulin administrationfor.

3. (canceled)

4. (canceled)

5. The method of claim 1, wherein the complication of hyperglycaemia is selected from the group consisting of a retinopathy, neuropathy, nephropathy, a cardiovascular injury, and lesions on the feet.

6. A method for reducing the weight, and/or preventing weight gain, andor preventing or treating obesity, andor reducing appetite, andor suppressing hunger in a subject in need thereof, said method comprising administering to the subject a pharmaceutical composition comprising an aminosteroid derivative of formula (I)
wherein: 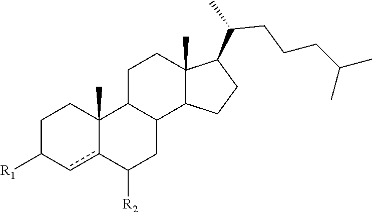
R₁ and R₂ are each, independently, a hydroxyl group or a polyamino chain of formula —NR₃R₄;.
R₃ is -(A-X)-A-NR₆R₇;.
each A, which may be identical or different, is an alkyl chain comprising 1 to 7 carbons, each carbon being independently optionally substituted with at least one alkyl, aryl or ester group;
each X, which may be identical or different, is an oxygen atom, an NR₅ group or a single bond;
each R₅ is independently a hydrogen atom, an alkyl group, an aryl group or an ester group;
R₆ and R₇ are each, independently, a hydrogen atom, an alkyl group, an aryl group or an ester group;
or R₆ and R₇ together with the N atom to which they are attached form a heterocyclyl group;
p is 1-4; and
R₄ is a hydrogen atom, an alkyl group, an aryl group or an ester group;
wherein at least one of R₁ and R₂ is a polyamino chain of formula —NR₃R₄, and a pharmaceutically acceptable carrier.

7. (canceled)

8. A method for improving the physical performance levels of a subject in need thereof, said method comprising administering to the subject a pharmaceutical composition comprising an aminosteroid derivative of formula (I) 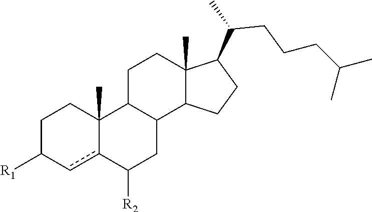
wherein:
R₁ and R₂ are each, independently, a hydroxyl group or a polyamino chain of formula —NR₃R₄;
R₃ is -(A-X)ₚ-A-NR₆R₇;
each A, which may be identical or different, is an alkyl chain comprising 1 to 7 carbons, each carbon being independently optionally substituted with at least one alkyl, aryl or ester group;
each X, which may be identical or different, is an oxygen atom, an NR₅ group or a single bond;
each R₅ is independently a hydrogen atom, an alkyl group, an aryl group or an ester group;
R₆ and R₇ are each, independently, a hydrogen atom, an alkyl group, an aryl group or an ester group;
or R₆ and R₇ together with the N atom to which they are attached form a heterocyclyl group;
p is 1-4; and
R₄ is a hydrogen atom, an alkyl group, an aryl group or an ester group;
wherein at least one of R₁ and R₂ is a polyamino chain of formula —NR₃R₄, and a pharmaceutically acceptable carrier.

9. An aminosteroid derivative of formula (II) 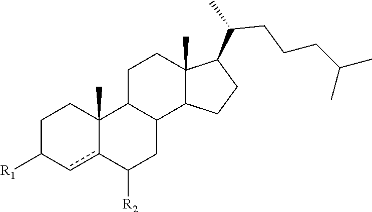
wherein:
R₁ and R₂ are each, independently, is chosen from a hydroxyl group mor a polyamino chain of formula —NR₃R₄;
R₂ is -(A-X)ₚ-A-NR₆R₇;
each A, which may be identical or different, is an alkyl chain comprising 1 to 7 carbons, each carbon being independently optionally substituted with at least one alkyl, aryl or ester group;
each X, which may be identical or different, is an oxygen atom, an NR_{S} group or a single bond;
each R₅ is independently a hydrogen atom, an alkyl group, an aryl group or an ester group;
R₆ and R₇ are each, independently, a hydrogen atom, an aryl group or an ester group;
or R₆ and R₇ together with the N atom to which they are attached form a heterocyclyl group;
p is 1-4; and
R₄ is a hydrogen atom, an alkyl group, an aryl group or an ester group; wherein at least one of R₁ and R₂ is a polyamino chain of formula —NR₃R₄, the bond in the form of a dashed line represents either a single bond or a double bond, with the proviso that if the bond in the form of a dashed line is a single bond and p is 1, then X is a single bond.

10. The aminosteroid derivative according to claim 9, in which R₂ is a polyamino chain of formula —NR₃R₄.

11. The aminosteroid derivative according to claim 9, wherein said aminosteroid derivative is selected from the group consisting of:
6β-(1,2-diamino ethane)cho lestan-3β-ol,
6β-diaminoprop ane)cho lestan-3β-ol,
6β-diaminobutane)cho lestan-3β-ol,
6β-diaminopentane)cholestan-3β-ol,
6β-diaminohexane)cholestan-3β-ol,
6β-diaminooctane)cholestan-3β-ol,
6β-diaminodecane)cholestan-3β-ol,
6β-(spermine)cholestan-3β-ol,
6β-bis(3-aminopropoxy)butane)cholestan-3β-ol,
6β-diaminododecane)cholestan-3β-ol,
6β-(3-aminopropyl)pyrrolidinone)cholestan-3β-ol,
6β-(3-aminopropyl)morpholine)cholestan-3β-ol,
6β-(3-aminopropyl)pyrrolidine)cholestan-3β-ol,
6β-(3-aminopropyl)imidazole)cholestan-3β-ol,
6β-(2-aminoallyl)piperazine)cholestan-3β-ol,
6β-(spermine)cholesten-3β-ol,
6β-(spermidine)cholesten-3β-ol,
3β,6β-bis(pentanediamine)cholest-3-ene,
3β,6β-bis(hexanediamine)cholest-3-ene,
3β,6β-bis(heptanediamine)cholest-3-ene, and
3β,6β-bis(octanediamine)cholest-3-ene.

12. The aminosteroid derivative according to claim 9, which aminosteroid derivative is 6β-(spermine)cholestan-3β-ol (ST1 0) or 6β-(spermidine)cholesten-3β-ol (ST20).

13. A pharmaceutical composition comprising the aminosteroid derivative of claim 9 and a pharmaceutically acceptable carrier.
